(19) United States
(12) Patent Application Publication
(10) Pub. No.: US 2026/0241076 A1
(43) Pub. Date: Aug. 20, 2026

(54) SYSTEM AND METHOD FOR THREE-DIMESIONAL CELL CULTURE WITH SPONGY-STRUCTURED HYDROGEL SCAFFOLD

(71) Applicant: National Yang Ming Chiao Tung University, Taipei City (TW)

(72) Inventors: Mu-Hsin CHANG, Taipei City (TW); Chi-Ying Huang, Taipei City (TW); Tai-Shan Cheng, Taipei City (TW); Yu-Tang Huang, Taipei City (TW)

(73) Assignee: National Yang Ming Chiao Tung University, Taipei City (TW)

(21) Appl. No.: 19/540,123

(22) Filed: Feb. 13, 2026

Related U.S. Application Data
(60) Provisional application No. 63/758,802, filed on Feb. 14, 2025.

(51) Int. Cl. A61L 27/52; A61K 38/00; A61L 27/26; A61L 27/54; A61L 27/56

(52) U.S. Cl. CPC A61L 27/52; A61K 38/00; A61L 27/26; A61L 27/54; A61L 27/56

(57) ABSTRACT

The present invention provides a spongy-structured hydrogel scaffold, comprising base components including (a) a structural protein, (b) an elastic-bridging molecule, and (c) a growth factor-binding molecule, which is constructed for three-dimensional (3D) cell culture, particularly tumoroids, providing improvement in structural integrity and uniformity of cell size, so as to enhance the accuracy and reliability of cancer research and drug efficacy assessments.

### FIELD OF THE INVENTION

The invention relates to a system and method for three-dimensional (3D) cell culture for biological cells, particularly tumoroids.

### BACKGROUND OF THE INVENTION

Preclinical experiments with animal models are time-consuming and raise ethical concerns, as emphasized by the 3Rs (Replacement, Reduction, and Refinement) alternative principle [1]. This discrepancy highlights the caution needed when extrapolating cell-based experimental results to broader medical applications. However, cancer cells are normally cultured in conventional two-dimensional (2D) plastic dishes, which lack physiological relevance and fail to recapitulate the complex tumor microenvironment, so as to limit their utility for preclinical anti-cancer drug screening.

Differential drug responses between 2D cell cultures and animal models, particularly in cancer patient-derived cells, emphasize the necessity for more representative models. To address these limitations, human ex vivo 3D cell cultures, particularly organoids derived from cancer patients'tumors, offer a promising solution. The advancement of 3D cell culture technology has profoundly impacted disease research, precision medicine, and drug development. In cancer research, where intra-tumor heterogeneity poses challenges such as metastasis, recurrence, and treatment resistance, the need for more accurate and personalized tumor models using 3D cell culture techniques has become increasingly apparent [2].

Although there are a few approaches for 3D cell culture, for example, GEcoll® ACD 3D Culture Kit or AcroCyte® R3CE 24-Well Plate. In the GEcoll®ACD 3D culture kit, a scaffold system based on alginate is used, wherein alginate is a natural polysaccharide from brown algael; but alginate lacks the RGD (Arg-Gly-Asp) peptide, essential for cell-ECM adhesion. The AcroCyte® R3CE 24-Well Plate is a pre-coated plate for culturing tumoroids, in which R3CE hinges on an optimized surface-coating procedure that applies a biocompatible polyelectrolyte multilayer (PEM) film. This film comprises alternating layers of poly-L-lysine (PLL) and poly-L-glutamic acid (PLGA), meticulously deposited onto polystyrene tissue culture plates (TCP). However, this approach is less effective in preserving the three-dimensional (3D) architecture of the cells, leading to disintegration and irregular cell sizes. The absence of a supporting scaffold resulted in poor structural integrity and inconsistent cellular morphology.

Therefore, it is desirable to develop a new scaffold for 3D cell culture.

### SUMMARY OF THE INVENTION

Accordingly, the present invention provides a system for three-dimensional (3D) cell culture can be constructed, which provides improvement in structural integrity and uniformity of cell size.

In one aspect, the present invention provides a spongy-structured scaffold for three-dimensional (3D) cell culture for biological cells, which is obtained by the method comprising the following steps:
(i) mixing base components including (a) a structural protein, (b) an elastic-bridging molecule, and (c) a growth factor-binding molecule, and then stirring them to obtain a base mixture;
(ii) adding a growth factor to the base mixture obtained in step (i) to obtain a hydrogel base;
(iii) placing the hydrogel base obtained in step (ii) at about 0° C. to 10° C. to form a liquid gel to prevent from premature gelation;
(iv) incubating the liquid gel obtained in step (iii) at about 20° C. to 50° C. to form a shape-fixed solid gel in a container; and
(v) subjecting to the solid gel obtained in step (iv) under a freeze-thaw procedure, including a freezing step at about 0° C. to −20° C. and subsequent a thawing step, to form a spongy-structured hydrogel having porous texture to obtain the scaffold for 3D cell culture; wherein the scaffold for 3D cell culture shall be equilibrated to room temperature before use.

In one embodiment of the invention, the cells are tumoroids.

In one embodiment of the invention, the growth factor is selected from the group consisting of transforming growth factor-beta (TGF-β), epidermal growth factor (EGF), insulin-like growth factor-1 (IGF-1), basic fibroblast growth factor (bFGF), platelet-derived growth factor (PDGF), vascular endothelial growth factor (VEGF), and nerve growth factor (NGF).

In one embodiment of the invention, the structural protein is selected from the group consisting of collagen, laminin, fibronectin, and fibrinogen.

In one embodiment of the invention, the elastic-bridging molecule is selected from the group consisting of entactin, elastin, heparan sulfate, and hyaluronic acid.

In one embodiment of the invention, the growth factor-binding molecule is a proteoglycan which is selected from the group consisting of heparan sulfate, chondroitin sulfate, and dermatan sulfate.

In a further aspect, the present invention provides a method for preparing a spongy-structured scaffold for 3D cell culture, comprising:
(i) mixing base components including (a) a structural protein, (b) an elastic-bridging molecule, and (c) a growth factor-binding molecule, and then stirring them to obtain a base mixture;
(ii) adding a growth factor to the base mixture obtained in step (i) to obtain a hydrogel base;
(iii) placing the hydrogel base obtained in step (ii) at about 0° C. to 10° C. to form a liquid gel to prevent from premature gelation;
(iv) incubating the liquid gel obtained in step (iii) at about 20° C. to 50° C. to form a shape-fixed solid gel in a container; and
(v) subjecting to the solid gel obtained in step (iv) under a freeze-thaw procedure, including a freezing step at about 0° C. to −20° C. and subsequent a thawing step, to form a spongy-structured hydrogel having porous texture to obtain the scaffold for 3D cell culture.

In one yet aspect, the present invention provides a system for three-dimensional (3D) cell culture for biological cells, wherein the system is composed of the spongy-structured hydrogel scaffold according to the invention.

According to the invention, the spongy-structured hydrogel scaffold has an internal structure that is controllably altered by a a freeze-thaw procedure., The hydrogel before a freezing step exhibits a dense, substantially uniform structural network; but upon exposure to at least one freeze-thaw cycle, the hydrogel undergoes microstructural reorganization and develops a plurality of interconnected porous microdomains. It is unexpected that these freeze-thaw procedure induces pores and forms a sponge like structure that provides physical and diffusional advantages facilitating the formation of compact, spherical cells, particularly cancer cells (i.e., tumoroids).

In the present invention, the cancer cells obtained by using the spongy-structured hydrogel scaffold exhibit enhanced invasion capacity, improved morphological uniformity, and increased structural completeness compared to the cancer cells obtained by culturing in the no hydrogel without undergoing the freeze-thaw procedure. The spongy-structured hydrogel scaffold provides altered porosity that enables controlled spatial confinement, promotes self-organization of cells into well-defined spheroids, and also improves nutrient supporting and/or drug penetrability. The system for 3D cell culture composed of spongy-structured hydrogel scaffold facilitates cell growth and enables the formation of 3D tumoroid by improving structural integrity and uniformity of the cell size of the cells as cultured.

In the present invention, the any growth factor required for and/or contributing to the culture of specific biological cells may be used to facilitate the cell growth and/or cell culture. Examples of the growth factor include is transforming growth factor-beta (TGF-β), epidermal growth factor (EGF), insulin-like growth factor-1 (IGF-1), basic fibroblast growth factor (bFGF), platelet-derived growth factor (PDGF), vascular endothelial growth factor (VEGF), and nerve growth factor (NGF).

In one embodiment of the present invention, the structural protein is selected from the group consisting of collagen, laminin, fibronectin, and fibrinogen.

In one embodiment of the present invention, the elastic-bridging molecule is selected from the group consisting of entactin, elastin, heparan sulfate, and hyaluronic acid.

In one embodiment of the present invention, the growth factor-binding molecule is a proteoglycan which is selected from the group consisting of heparan sulfate, chondroitin sulfate, and dermatan sulfate.

In one particular example of the invention, In one particular example of the invention, the base components of the hydrogel comprises (1) laminin; (2) collagen; and (3) entactin; wherein (1) laminin, having a proportion at about 50-70% of the total protein content provides structural integrity and facilitates cell adhesion; (2) collagen, having a proportion at about 20-40%, provides the matrix framework and enhances the mechanical stability; and (3) entactin, having a proportion at about 5-10%, acts as a bridging molecule stabilizing interaction between laminin and collagen to ensure structural organization.

In a yet aspect, the present invention provides a system for screening a drug against a cancer using the spongy-structured hydrogel scaffold for cell culture, which comprises the spongy-structured hydrogel scaffold, in which caner cells are cultured.

In some examples of the present invention, the cancer cells are selected from the group consisting of pancreatic cancer patient-derived cancer cells, human pancreatic stellate cells (hPSC), PANC-1 (originating from a pancreatic duct carcinoma in a 56-year-old Caucasian male), DLD-1 (which is a colorectal adenocarcinoma cell line isolated from the large intestine of a colon adenocarcinoma patient), HSC3 (which is a human oral squamous carcinoma cell line), and FaDu cells (which is a cell line isolated from a hypopharyngeal tumor of a squamous cell carcinoma patient).

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the method of preparing the spongy hydrogel.

FIG. 2 shows a comparison between non-frozen hydrogel and the spongy-structured hydrogel obtained according to the invention.

FIGS. 3A-3B show a comparison of different 3D culturing methods for culturing PANC-1 cells. FIG. 3A shows the images of morphology presented by PANC-1 cell by using spongy hydrogel, GEcoll® ACD 3D Culture Kit, and AcroCyte® R3CE 24-Well Plate. FIG. 3B shows the diameters of tumoroids cultured using different methods over various days. *p<0.05, **p<0.01, (n=5).

FIGS. 4A-4B show a comparison of the cancer cell cultures in different structured hydrogels. FIG. 4A shows a comparison of PANC-1 tumoroids formation ability by culturing using non-frozen and frozen hydrogel. FIG. 4B shows a comparison of cancer cell culture in the both hydrogels.

FIG. 5 shows that various cancer cell lines could be successfully cultured in the spongy-structured hydrogel scaffold and form into 3D tumoroids.

FIGS. 6A-6B show the establishment of a system for cell culture, including PANC-1 with human pancreatic stellate cells (hPSC) in the spongy-structured hydrogel scaffold. FIG. 6A shows the illustration of the co-culture system. FIG. 6B shows the images of hPSC inducing the progression of PANC-1 tumoroids.

FIG. 7 shows that the system could be used for screening an anti-cancer drug against gemcitabine, providing a comparison between the drug response in the system for co-culturing patient-derived primary cells (SP-1) and hPSC, and the system for culturing SP-1 alone.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present disclosure, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an” and “the” include plural references unless explicitly indicated otherwise. Thus, for example, reference to “a sample” includes a plurality of such samples and their equivalents known to those skilled in the art.

The term “comprise” or “comprising” is generally used in the sense of include/including, which means permitting the presence of one or more features, ingredients, or components. The term “comprise” or “comprising” encompasses the term “consists” or “consisting of.”

As used herein, the term “hydrogel” refers to a network of hydrophilic polymer chains that can absorb and retain substantial amounts of water while maintaining a distinct structure. Hydrogel is a highly water-absorbent material consisting of a three-dimensional network of polymer chains that can maintain a gel state after absorbing water and can be less likely to flow or leak. The materials of preparing the hydrogel are often biocompatible and tunable, making them suitable for a range of applications in biomedical and pharmaceutical fields, such as drug delivery, tissue engineering, and wound care. In the present invention, the hydrogel composition is used for cell culturing, especially for culturing 3D tumoroids.

The present invention provides a method for preparing a scaffold for three-dimensional (3D) cell culture for biological cells, particularly tumoroids, which comprises the following steps:
(i) mixing base components including (a) a structural protein, (b) an elastic-bridging molecule, and (c) a growth factor-binding molecule, and then stirring them to obtain a base mixture;
(ii) adding a growth factor to the base mixture obtained in step (i) to obtain a hydrogel base;
(iii) placing the hydrogel base obtained in step (ii) at about 0° C. to 10° C. to form a liquid gel to prevent from premature gelation;
(iv) incubating the liquid gel obtained in step (iii) at about 20° C. to 50° C. to form a shape-fixed solid gel in a container; and
(v) exposing the solid gel obtained in step (iv) to a freezing step at about 0° C. to −20° C. and subsequent a thawing step to form a spongy-structured hydrogel having porous texture to obtain the scaffold for 3D cell culture;
   wherein the scaffold for 3D cell culture shall be equilibrated to room temperature before use.

In the present invention, a spongy-structured hydrogel scaffold is obtained, which refers to a scaffold made from a hydrogel material characterized by a porous, sponge-like architecture, typically consisting of interconnected cavities or channels. This structure enhances fluid absorption, diffusion, and mechanical flexibility, making it suitable for applications such as scaffolding, drug delivery, and/or tissue engineering.

According to the present invention, the spongy-structured hydrogel scaffold is featured in its specialized structure generated through a freeze-thaw procedure, the porous texture, which enables thespongy-structured hydrogel scaffold mimics the extracellular matrix (ECM) in order to promote the growth of the 3D tumoroids and improve structural integrity and uniformity of cell size, thereby enhancing the accuracy and reliability of cancer research and drug efficacy assessments.

According to the present invention, the method of preparing thespongy-structured hydrogel scaffold comprises the steps of:
(i) mixing base components comprising (a) a structural protein base, (b) an elastic-bridging molecule, and (c) a growth factor-binding molecule in a container at about 0 to 10° C., and then stirring them without bubble formation to obtain a base mixture;
(ii) adding growth factors to the base mixture obtained in step (i) to obtain a hydrogel base;
(iii) placing the hydrogel base obtained in step (ii) at about 0° C. to 10° C. to form a liquid gel to prevent from premature gelation;
(iv) incubating the liquid gel obtained in step (iii) at about 20° C. to 50° C. to form a shape-fixed solid gel in a container; and
(v) freezing the solid gel obtained in step (iv) at about 0° C. to −20° C. to form aspongy-structured hydrogel scaffold having porous texture.

### Hydrogel Composition of Base Components

Thespongy-structured hydrogel scaffold is made from a hydrogel composition, which comprises at least one biocompatible polymeric material capable of forming a crosslinked three-dimensional network. Although the specific formulation may vary, suitable materials include natural polymers, synthetic polymers, ECM-mimetic proteins, or combinations thereof. The hydrogel is capable of maintaining structural stability under physiological culture conditions and supporting long-term cell viability. In the present invention, the hydrogel composition comprises (a) a structural protein, (b) an elastic-bridging molecule, and (c) a growth factor-binding molecule.

### Structural Protein

The structural protein contributes to tensile strength, structural integrity, and mechanical stability within the matrix of hydrogel. Examples of structural protein include collagen (e.g., Types I, III, and IV), laminin, fibronectin, and fibrinogen. Collagen provides a fibrous framework, while laminin and fibronectin act as primary adhesion molecules, enhancing cell attachment and ensuring the matrix's structural cohesiveness.

### Elastic-Bridging Molecule

The elastic-bridging molecule ensures elasticity and structural stability of hydrogel. Examples of elastic-bridging molecule include entactin (also known as nidogen), elastin, heparan sulfate, and hyaluronic acid. The elastic-bridging molecule is able to bridge interaction between the structural protein such as laminin and collagen, enabling the hydrogel proceed structural re-organization.

### Growth Factor-Binding Molecule

The growth factor-binding molecule plays a crucial role not only in facilitating the binding of growth factors within the hydrogel matrix but also in cellular signaling, cell proliferation, and maintaining matrix hydration and structure. Examples of growth factor-binding molecule include proteoglycans such as heparan sulfate, chondroitin sulfate, and dermatan sulfate.

### Growth Factor

The growth factor in the hydrogel can help stimulating the cellular activity, such as cell proliferation, adhesion, and differentiation, fostering a biologically active environment suitable for 3D cell development. Examples of growth factor include transforming growth factor-beta (TGF-β), epidermal growth factor (EGF), insulin-like growth factor-1 (IGF-1), basic fibroblast growth factor (bFGF), platelet-derived growth factor (PDGF), vascular endothelial growth factor (VEGF), and nerve growth factor (NGF).

In one particular example of the present invention, the spongy-structured hydrogel scaffold is made from a hydrogel composition comprising (1) laminin, having a proportion at about 50-70% of the total protein content, and providing structural integrity and facilitates cell adhesion; (2) collagen, having a proportion at about 20-40%, and providing the matrix framework and enhancing the mechanical stability; and (3) entactin, having a proportion at about 5-10%, which acts as a bridging molecule stabilizing interaction between laminin and collagen to ensure structural organization.

In one example of the present invention, heparan sulfate is incorporated for binding and regulating growth factors, supporting cellular signaling pathways which are critical for proliferation and differentiation.

In one example of the present invention, thespongy-structured hydrogel scaffold contains growth factors, such as TGF-β, EGF, IGF-1, and bFGF in an effective amount to stimulate cellular activity.

According to the invention, the base components of the hydrogel composition are mixed to obtain a base mixture. The base mixture of the three base components remains in liquid form at 4° C. because low energy at this temperature prevents protein bonding needed for gel formation. Gelation begins at 10-15° C. and reaches a stable, fully gelled state at 37° C., forming a matrix that mimics the in vivo environment. During the process of formation, the hydrogel is not returned to a 4° C. state. However, according to experimental observations, freezing the hydrogel at −20° C. does not revert it into liquid form again, so as to obtain a spongy-structured hydrogel scaffold, which creats an optimal environment for tumoroids growth and modeling complex tumor microenvironments.

In the present invention, the spongy-structured hydrogel scaffold is characterized in that its water content crystallizes during the freezing process, small ice crystals are formed togive a sponge-like porous texture. Once thawed, the gel develops numerous internal cavities. This porous texture can mimic native microenvironments ex vivo, resembling the cancellous bone architecture in bone marrow, the parenchyma in the lung, or other reticuloendothelial systems in the human body. Thespongy-structured hydrogel scaffold preserves the complex composition and architecture of the ECM, closely replicating the tumor microenvironment found in native human tissues. Within this porous texture, cancer cells can grow effectively in a 3D environment, facilitating the formation of 3D tumoroids and making the hydrogel performed as an ideal medium for cultivating 3D tumoroids ex vivo.

According to the present invention, the spongy-structured hydrogel scaffold is suitable for coating on any standard tissue culture plate without the need for special materials, offering a broad applicability.

According to the present invention, the spongy-structured hydrogel scaffold successfully mimics the tumor microenvironment which not only yields a higher success rate but also offers superior growth rates and cellular characteristics.

In the invention, the hydrogel composition undergos a freeze-thaw procedure, to obtain the spongy-structured hydrogel scaffold. As compared to a non-frozen spongy hydrogel, the spongy-structured hydrogel scaffold significantly facilitates cell growth within the interstices, enabling the formation of 3D tumoroids.

In the invention, the spongy-structured hydrogel scaffold presents a wonderful compatibility with multiple cell lines, including PANC-1, DLD-1, HSC3, and FaDu, and patient-derived primary tumor cells (3R-4 and 3R-5).

In the invention, human pancreatic stellate cells (hPSC) can induce the progression of PANC-1 tumoroids when the two types of cells were co-cultured in the spongy-structured hydrogel scaffold.

It is also ascertained in the example that the drug response to gemcitabine was lower in the patient-derived primary cells (SP-1)/hPSC co-culture system. The 3D tumoroid model using the spongy-structured hydrogel scaffold effectively mimics the fibrotic behavior of pancreatic cancer, which is resistant to the standard anti-cancer drug gemcitabine, as observed in vivo.

Thespongy-structured hydrogel scaffold provides not only an excellent model for cancer drug development and patient drug screening, but also holds promise for future clinical applications in anti-cancer drug screening for cancer patients, particularly in enhancing the effectiveness and precision of therapeutic interventions.

The present invention is further illustrated by the following examples, which are provided for the purpose of demonstration rather than limitation.

### Methods

### Freeze-Thaw Procedure for Structural Modulation

The hydrogel comprising was undergoing a freeze-thaw procedure to induce microstructural reorganization. The hydrogel possesses a dense internal morphology characterized by restricted pore spaces and limited heterogeneity. The hydrogel was subjected to at least a freeze-thaw procedure, so that the aqueous phase of the hydrogel forms ice crystals that mechanically reorganize the polymeric network; and when thawing, discrete porous microstructures remain, resulting in a spongy, interconnected internal framework.

### Cell Culture in the Spongy-Structured Hydrogel Scaffold

A broad range of biological cells may be cultured in the spongy-structured hydrogel scaffold. Particularly, cancer cells, including but not limited to pancreatic, colorectal, lung, or head-and-neck carcinoma cells are seeded into the hydrogel. Cells may be taken by direct encapsulation, surface seeding followed by infiltration, or perfusion-based loading to be cultured in the spongy-structured hydrogel scaffold.

### Tumoroid Formation Behavior

Cancer cells cultured within the spongy-structured hydrogel scaffold exhibited accelerated aggregation and formed compact, spherical tumoroids with well-defined boundaries and uniform structural organization. The porous microarchitecture was generated through a freeze-thaw procedure to obtain the spongy-structured hydrogel scaffold, which enhanced intercellular contact and facilitated controlled spatial confinement, resulting in spheroids with consistent morphology and improved structural integrity across the culture.

As a comparison, cancer cells were grown in a non-frozen hydrogel having irregular and loosely associated aggregates. The denser, non-porous matrix impeded coordinated cell-cell assembly, yielding spheroid-like structures with poor compaction, indistinct borders, and reduced formation efficiency. It was observed in the examples that the porosity induced through the freeze-thaw procedure directly improved tumoroid uniformity and maturation as compared to the non-frozen hydrogel.

### EXAMPLES

### Example 1 Preparation of the Spongy-Structered Hydrogel Scaffold

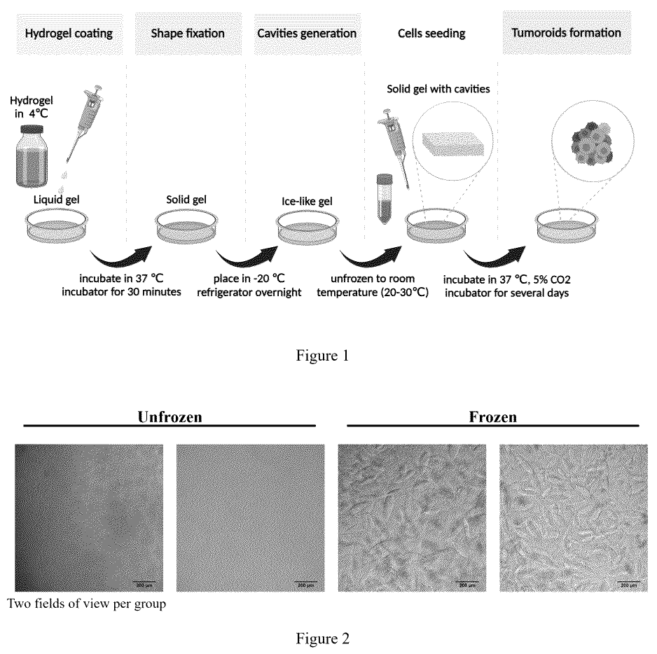

As shown in FIG. 1, the method of preparing thespongy-structured hydrogel scaffold was provided.

Initially, all the base components of the hydrogel, including heparan sulfate, laminin, collagen, and entactin, were thawed on ice to maintain their integrity. The base components were then combined in a cold, sterile container and gently stirred to avoid bubble formation to form a mixture. Growth factors including EGF and bFGF were incorporated into the mixture, which was gently mixed to ensure even distribution. The mixture was kept on ice as a liquid gel to prevent premature gelation. Afterwards, the liquid gel was placed in 37° C. to form a shape-fixed solid gel in the container. The solid gel was then frozen at −20° C. to form the final product, called as the spongy-structured hydrogel scaffold, which is characterized by porous texture. Thespongy-structured hydrogel scaffold was then used for cell culture or 3D tumoroids formation.

### Example 2 Comparison Between Frozen and Non-Frozen Hydrogels

As shown in FIG. 2, a hydrogel sample was maintained at standard temperature to represent the non-frozen condition for comparison (called as “a non-frozen hydrogel”). The other sample was subjected to a freeze-thaw procedure, which comprises at least one freeze-thaw cycle. It was confirmed by microscopy that the non-frozen hydrogel exhibited a dense and substantially homogeneous internal matrix, but the hydrogel through a freeze-thaw procedure displayed numerous micro-porous cavities dispersed throughout the network.

### Example 3 Study of Different Components of Hydrogels

A comparative study was conducted using three different methods to culture PANC-1 cells into 3D tumoroids. The first method utilized the GEcoll® ACD 3D culture kit, which employs a scaffold system based on alginate, a natural polysaccharide derived from brown algae. The alginate lacks the RGD (Arg-Gly-Asp) peptide, which is crucial for cell-ECM adhesion. Therefore, gelatin, derived from connective tissue collagen and containing RGD sequences, was added to enhance cell adhesion by interacting with integrins on the cell membrane. The second method utilized the AcroCyte® R3CE 24-Well Plate, a pre-coated plate designed for culturing cancer tumoroids. The R3CE method relies on an optimized surface-coating procedure that applies a biocompatible polyelectrolyte multilayer (PEM) film, which is composed of alternating layers of poly-L-lysine (PLL) and poly-L-glutamic acid (PLGA), carefully deposited onto polystyrene tissue culture plates (TCP). The third method utilized thespongy-structured hydrogel scaffold provided by the present invention. Bothspongy-structured hydrogel scaffold and GEcoll® ACD 3D Culture Kit are scaffold-based.

PANC-1 cells were seeded at a density of 1000 cells per well and cultured at 37° C. for 5 to 12 days.

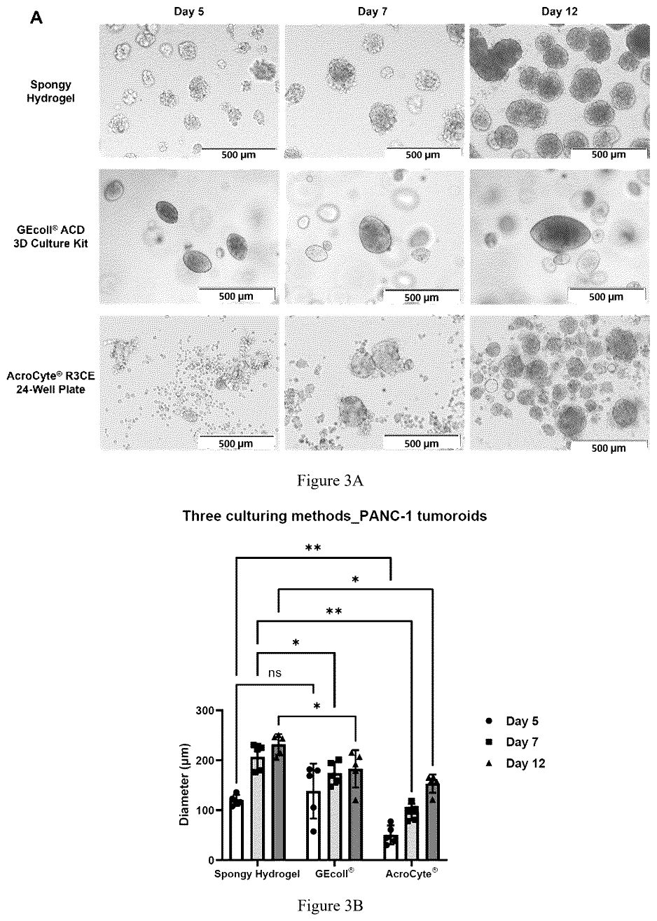

As shown in FIGS. 3A-3B, three methods successfully formed 3D tumoroids, but significant differences in cell morphology were observed. Tumoroids cultured by using these methods exhibited a spherical shape with well-defined cell boundaries. However, the diameter of tumoroids presented an obvious change by culturing with spongy hydrogel, which showed a faster growth rate and more consistent cell size compared to GEcoll® ACD 3D Culture Kit. This suggests that the spongy-structured hydrogel scaffold not only provided an ECM-scaffold to maintain cellular structure, but also ensured structural integrity and uniformity of cell size within its specialized structural pores. In contrast, AcroCyte® R3CE 24-Well Plate, a scaffold-free approach, was less effective in maintaining the 3D structure of the cells (FIG. 3A). After several days of culture, the cells exhibited disintegration and irregular cell size. The lack of a scaffold resulted in poor structural integrity and inconsistent cell size (FIG. 3B). Comparing these three methods highlights the advantages of spongy hydrogel. The result of culturing 3D cells in thespongy-structured hydrogel scaffold proves that it successfully mimicked the tumor microenvironment which not only yielded a higher success rate but also offered superior growth rates and cellular characteristics, making it become a most suitable material for cancer drug development among the three tested materials.

### Example 4 Study of Tumoroids Formation Ability on Frozen and Non-Frozen Hydrogels

A comparative evaluation experiment of PANC-1 tumoroids formation ability on the non-frozeb hydrogel and the spongy-structured hydrogel scaffold was conducted. The two sets of hydrogels were conducted by coating 200 μL liquid hydrogel in separate 24-well plates. One plate remained in unfrozen condition, and the other underwent a freeze-thaw procedure. PANC-1 cells were seeded at a density of 1000 cells per well and cultured at 37° C. for 7 days.

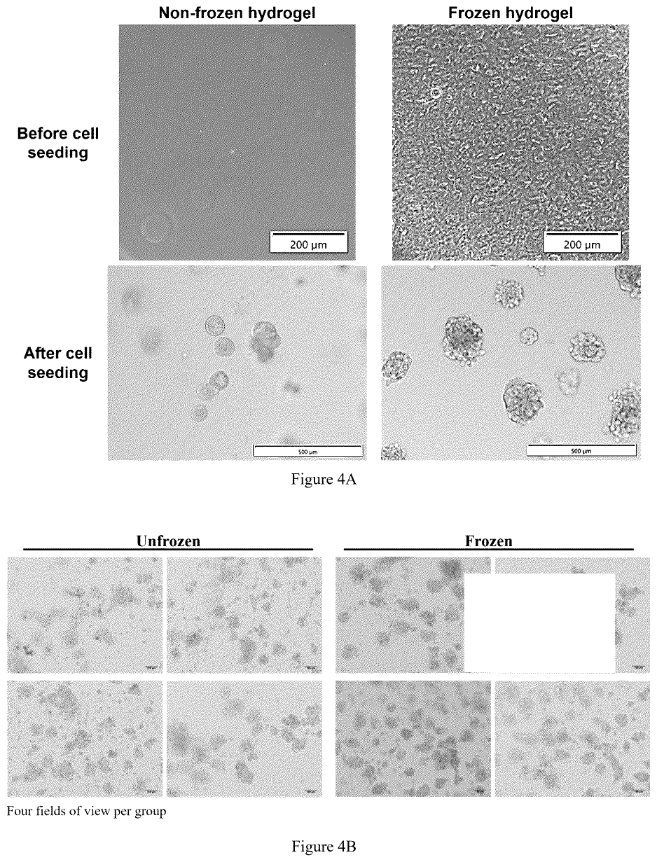

As shown in FIG. 4A, the non-frozen hydrogel was observed to be lack of pores, hindered cell penetration into the ECM structure, making it challenging to cultivate 3D tumoroids. In contrast, the spongy-structured hydrogel scaffold through a freeze-thaw procedure, which filled with porous texture, mimicked the environment of human body cavities, facilitated cell growth within the interstices, enabling the formation of 3D tumoroids.

As shown in FIG. 4B, over the culture period, the cancer cells are cultured in the non-frozen hydrogel and the spongy-structured hydrogel scaffold through a freeze-thaw procedure rapidly formed compact tumoroids with well-defined spherical geometry. The cancer cells in the non-frozen hydrogel formed dispersed aggregates lacking structural uniformity. These results demonstrate that porosity generated through freeze-thaw procedure enhances spheroid formation efficiency.

### Example 5 Assessment of the Spongy-Structured Hydrogel Scaffold for Culturing Multiple Cell Lines

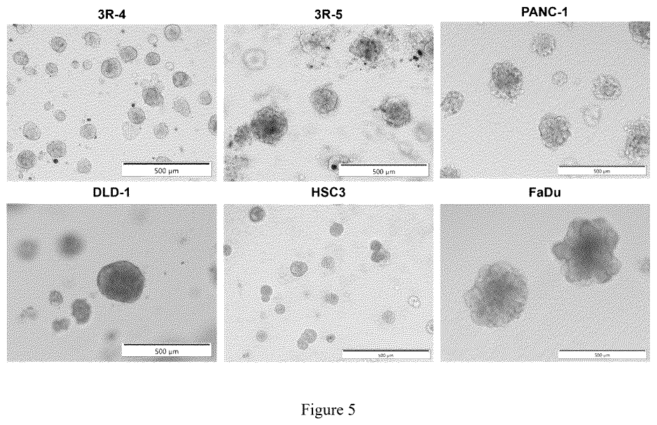

Two pancreatic cancer patient-derived cancer cells (3R-4 and 3R-5) and four cancer cell lines (PANC-1, DLD-1, HSC3, and FaDu) were seeded at a density of 1000 cells per well onthe spongy-structured hydrogel scaffold pre-coating 24-well plate and were cultured for 7 days. As shown in FIG. 5, the cultivation of 3D tumoroids usingspongy-structured hydrogel scaffold was successfully demonstrated in the all cancer cell lines and patient-derived primary cancer cells. All cell lines(types) also presented a wonderful structural integrity and uniformity of cell sizes.

### Example 6 Co-Culture Test

Pancreatic cancer cells, which are characterized by aggressive, metastatic, and resilient traits, typically exhibit a notably dense and fibrous stroma. A system for co-culturing human pancreatic stellate cells (hPSC) with PANC-1 cells was created using the spongy-structured hydrogel scaffold, for purpose of simulating the tumor microenvironment of pancreatic cancer within the human body.

A direct co-culture test in the spongy-structured hydrogel scaffold was conducted to simulate the tumor microenvironment of pancreatic cancer. hPSC and PANC-1 cells were co-cultured at a ratio of 1:1. The co-cultured cells were maintained under standard cell culture conditions. Images were captured using phase-contrast microscopy to assess the structural characteristics of the tumoroids and were taken on day 3 and day 7 to monitor the progression of cell growth and morphological changes.

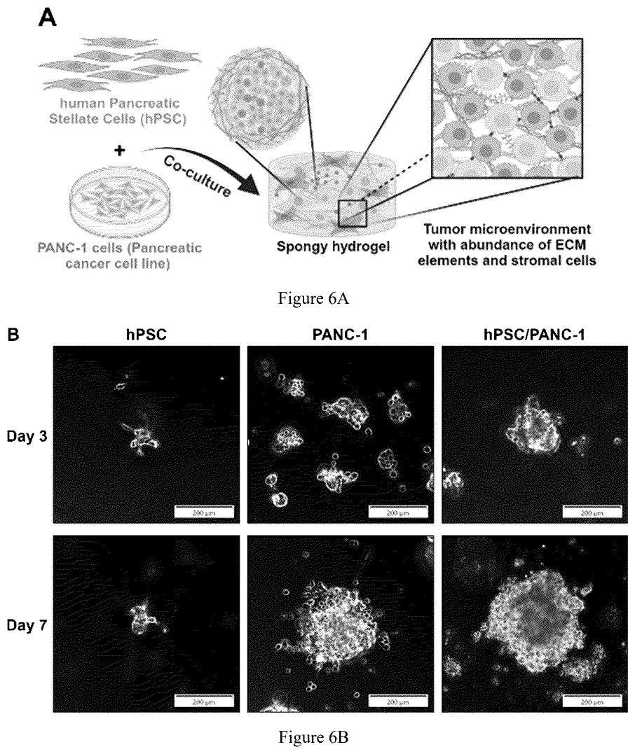

FIG. 6A illustrates an authentic simulation of the in vivo environment by utilizing thespongy-structured hydrogel scaffold for the co-culture of the two types of cells.

As shown in FIG. 6B, the cancer cells were co-cultured with stellate cells in the spongy-structured hydrogel scaffold, and the co-cultured cells potentially exhibited better growth and more solid morphology as compared with the system for culturing PANC-1 tumoroids alone.

### Example 7 Cell Viability Assay

Additional experiments were conducted to assess the efficacy in enhancing tumor growth and replicating known drug resistance behaviors observed in vivo, using the spongy-structured hydrogel scaffold mimicking the tumor microenvironment through assessing the cancer cell viability. Specifically, pancreatic cancer is characterized by a dense, fibrotic stroma that creates a physical barrier and biochemical environment limiting drug penetration and efficacy.

To assess cell viability within the drug-treated 3D tumoroids, the CellTiter-Glo® 3D Cell Viability Assay was employed. This luminescent assay measured ATP levels, serving as an indicator of metabolically active cells, and provided a quantitative assessment of cell viability. The assay was conducted following the manufacturer's protocol, and luminescence was measured using a plate reader. The results were then compared between co-cultured tumoroids and PANC-1 tumoroids cultured alone after chemotherapy drug treatment. This allowed for an evaluation of the stromal cell interaction's effect on cancer cell behavior and drug resistance.

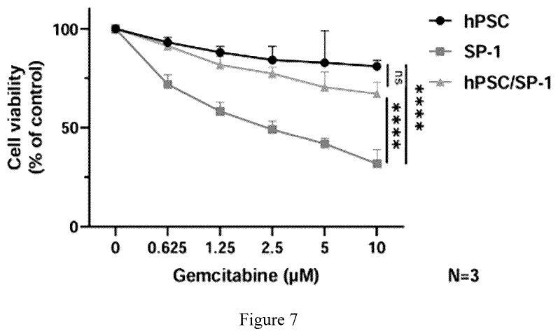

As shown in FIG. 7, drug response of gemcitabine was lower in patient-derived primary cells (SP-1)/hPSC co-culture system as compared to SP-1 alone. The result indicated that the 3D tumoroid model cultured in the spongy-structured hydrogel scaffold effectively mimicked fibrotic behavior of pancreatic cancer which was resistant to standard anti-cancer drugs gemcitabine observed in vivo. The spongy-structured hydrogel scaffold enabled the cultured 3D tumoroid to exhibit reduced sensitivity to gemcitabine, paralleling clinical drug resistance. The spongy-structured hydrogel scaffold successfully offered a supportive microenvironment conducive to tumor progression and captured the ECM-driven drug resistance mechanisms typical of pancreatic cancer.

Therefore, it is ascertained that the spongy-structured hydrogel scaffold is suitable for application in drug testing to identify therapies against fibrotic tumors, with the ultimate aim of advancing anti-cancer drug screening for pancreatic cancer patients. Additionally, by fully replicating a 3D tumor growth model in vitro, the spongy-structured hydrogel scaffold presents a significant advantage in reducing the need for animal testing. This approach not only supports ethical research practices but also provides a more physiologically relevant platform for evaluating drug efficacy in fibrotic tumor environments, potentially accelerating the development of targeted treatments.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

### REFERENCES

1. Rodrigues, D. B., R. L. Reis, and R. P. Pirraco, Modelling the complex nature of the tumor microenvironment: 3D tumor spheroids as an evolving tool. Journal of Biomedical Science, 2024. 31(1): p. 13.
2. Porter, R. J., G. I. Murray, and M. H. McLean, Current concepts in tumour-derived organoids. Br J Cancer, 2020. 123(8): p. 1209-1218.
3. Suenaga, H., et al., Phenotypic screening system using three-dimensional (3D) culture models for natural product screening. The Journal of Antibiotics, 2021. 74(10): p. 660-666.
4. Abuwatfa, W. H., W. G. Pitt, and G. A. Husseini, Scaffold-based 3D cell culture models in cancer research. Journal of Biomedical Science, 2024. 31(1): p. 7.
5. Li, Y. and E. Kumacheva, Hydrogel microenvironments for cancer spheroid growth and drug screening. Sci Adv, 2018. 4(4): p. eaas8998.
6. Yang, C.-R., et al., A Novel 3D Culture Scaffold to Shorten Development Time for Multicellular Tumor Spheroids. International Journal of Molecular Sciences, 2022. 23(22): p. 13962.
7. Tsai, H.-A., et al., Selection, Enrichment, and Maintenance of Self-Renewal Liver Stem/Progenitor Cells Utilizing Polypeptide Polyelectrolyte Multilayer Films. Biomacromolecules, 2010. 11(4): p. 994-1001.
8. Kozlova, N., et al., The Interplay of the Extracellular Matrix and Stromal Cells as a Drug Target in Stroma-Rich Cancers. Trends Pharmacol Sci, 2020. 41(3): p. 183-198.
9. Amrutkar, M., et al., Establishment and Characterization of Paired Primary Cultures of Human Pancreatic Cancer Cells and Stellate Cells Derived from the Same Tumor. Cells, 2020. 9(1): p. 227.

What is claimed is:

1. A spongy-structured scaffold for three-dimensional (3D) cell culture for biological cells, which is obtained by the method comprising the following steps:
(i) mixing base components including (a) a structural protein, (b) an elastic-bridging molecule, and (c) a growth factor-binding molecule, and then stirring them to obtain a base mixture;
(ii) adding a growth factor to the base mixture obtained in step (i) to obtain a hydrogel base;
(iii) placing the hydrogel base obtained in step (ii) at about 0° C. to 10° C. to form a liquid gel to prevent from premature gelation;
(iv) incubating the liquid gel obtained in step (iii) at about 20° C. to 50° C. to form a shape-fixed solid gel in a container; and
(v) subjecting to the solid gel obtained in step (iv) under a freeze-thaw procedure, including a freezing step at about 0° C. to −20° C.and subsequent a thawing step, to form a spongy-structured hydrogel having porous texture to obtain the scaffold for 3D cell culture;
wherein the scaffold for 3D cell culture shall be equilibrated to room temperature before use.

2. The spongy-structured scaffold of claim 1, in which the cells are tumoroids.

3. The spongy-structured scaffold of claim 1, which further comprises a growth factor which is selected from the group consisting of transforming growth factor-beta (TGF-β), epidermal growth factor (EGF), insulin-like growth factor-1 (IGF-1), basic fibroblast growth factor (bFGF), platelet-derived growth factor (PDGF), vascular endothelial growth factor (VEGF), and nerve growth factor (NGF).

4. The spongy-structured scaffold of claim 1, wherein the structural protein is selected from the group consisting of collagen, laminin, fibronectin, and fibrinogen.

5. The spongy-structured scaffold of claim 1, wherein the elastic-bridging molecule is selected from the group consisting of entactin, elastin, heparan sulfate, and hyaluronic acid.

6. The spongy-structured scaffold of claim 1, wherein the growth factor-binding molecule is a proteoglycan which is selected from the group consisting of heparan sulfate, chondroitin sulfate, and dermatan sulfate.

7. The spongy-structured scaffold of claim 1, wherein the base components comprise (1) laminin (2) collagen; and (3) entactin.

8. The spongy-structured scaffold of claim 7, wherein the base components comprise in a proportion of the total protein content, (1) laminin at about 50-70%, (2) collagen at about 20-40%, and (3) entactin at about 5-10%.

9. A method for preparing a spongy-structured scaffold for 3D cell culture, comprising:
(i) mixing base components including (a) a structural protein, (b) an elastic-bridging molecule, and (c) a growth factor-binding molecule, and then stirring them to obtain a base mixture;
(ii) adding a growth factor to the base mixture obtained in step (i) to obtain a hydrogel base;
(iii) placing the hydrogel base obtained in step (ii) at about 0° C. to 10° C. to form a liquid gel to prevent from premature gelation;
(iv) incubating the liquid gel obtained in step (iii) at about 20° C. to 50° C. to form a shape-fixed solid gel in a container; and
(v) subjecting to the solid gel obtained in step (iv) under a freeze-thaw procedure, including a freezing step at about 0° C. to −20° C. and subsequent a thawing step, to form a spongy-structured hydrogel having porous texture to obtain the scaffold for 3D cell culture;

10. The method of claim 9, which comprises:
(i) mixing base components including (a) a structural protein, (b) an elastic-bridging molecule, and (c) a growth factor-binding molecule in a container at about 0° C. to 10° C., and then stirring them without bubble formation to obtain a base mixture;
(ii) adding a growth factor to the base mixture obtained in step (i) to obtain a hydrogel base;
(iii) placing the hydrogel base obtained in step (ii) at about 0° C. to 10° C. to form a liquid gel to prevent from premature gelation;
(iv) incubating the liquid gel obtained in step (iii) at about 20° C. to 50° C. to form a shape-fixed solid gel in a container; and
(i) subjecting to the solid gel obtained in step (iv) under a freeze-thaw procedure, including a freezing step at about 0° C. to −20° C. and subsequent a thawing step, to
form a spongy-structured hydrogel having porous texture to obtain the scaffold for 3D cell culture.

11. The method of claim 9, wherein the structural protein is selected from the group consisting of collagen, laminin, fibronectin, and fibrinogen.

12. The method of claim 9, wherein the elastic-bridging molecule is selected from the group consisting of entactin, elastin, heparan sulfate, and hyaluronic acid.

13. The method of claim 9, wherein the growth factor-binding molecule is a proteoglycan which is selected from the group consisting of heparan sulfate, chondroitin sulfate, and dermatan sulfate.

14. The method of claim 9, wherein the growth factor is selected from the group consisting of transforming growth factor-beta (TGF-β), epidermal growth factor (EGF), insulin-like growth factor-1 (IGF-1), basic fibroblast growth factor (bFGF), platelet-derived growth factor (PDGF), vascular endothelial growth factor (VEGF), and nerve growth factor (NGF).

15. A system for three-dimensional (3D) cell culture for biological cells, wherein the system is composed of the spongy-structured hydrogel scaffold set forth in claim 1.

16. The system of claim 15, which is for co-culturing two or more cell lines.

17. The system of claim 15, which has function in facilitating cell growth and enabling the formation of 3D cell culture by improving structural integrity and uniformity in cell size,

18. The system of claim 15, wherein the cells are tumoroids.

19. The system of claim 15, the cells are selected from the group consisting of pancreatic cancer patient-derived cancer cells, human pancreatic stellate cells (hPSC), PANC-1, DLD-1, HSC3, and FaDu cells.
